(19) United States
(12) Patent Application Publication
(10) Pub. No.: US 2026/0165893 A1
(43) Pub. Date: Jun. 18, 2026

(54) BED WITH ADJUSTABLE ELEVATION PROFILE FOR CHANGING SLEEPING POSITION TO REDUCE SNORING AND SLEEP APNEA

(71) Applicant: Dazhai HUANG, Irvine, CA (US)

(72) Inventor: Dazhai HUANG, Irvine, CA (US)

(21) Appl. No.: 19/411,176

(22) Filed: Dec. 6, 2025

Related U.S. Application Data
(60) Provisional application No. 63/733,842, filed on Dec. 13, 2024.

(51) Int. Cl. A61G 7/00; A61B 5/00

(52) U.S. Cl. CPC A61G 7/001; A61B 5/4818; A61B 2562/0204; A61B 2562/0247

(57) ABSTRACT

A bed is equipped with two rollers mounted on a support frame, oriented parallel to the longitudinal direction of the mattress, located near two edges of the mattress and above the top surface of the mattress. A fabric sheet is rolled on the two rollers and loosely suspended between the two rollers across the mattress, and drapes down from the rollers and rests flat on the mattress in a center region of the mattress. During sleeping, when a snoring sound is detected, the rollers are controlled to rotate to pull the fabric sheet in an outward direction. This creates a slopped profile of a part of the fabric sheet, which gently pushes the user to help them turn to their sides.

### BACKGROUND OF THE INVENTION

This invention relates to a bed and related accessories, and in particular, it relates to a bed and accessories for changing a user's sleeping positions to reduce snoring and sleep apnea.

Snoring and sleep apnea can lead to various health problems. The onset and severity of snoring are often related to the sleeping position; for example, people tend to snore less when sleeping on their sides as compared to on their backs. Beds with air mattresses designed to reduce snoring by changing the users'sleeping position have been disclosed.

### SUMMARY OF THE INVENTION

The present invention is directed to a bed including accessories that can help the user change their sleeping position to reduce sleep disruption and improve sleep quality.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve the above objects, the present invention provides a bed which includes: a mattress having a flat top surface configured to receive a user's body; a support frame; two rollers mounted on the support frame, oriented parallel to a longitudinal direction of the mattress, located near two side edges of the mattress and above the top surface of the mattress; one or two motors configured to drive the rollers to rotate independently of each other; and a fabric sheet rolled on the two rollers and loosely suspended between the two rollers across the mattress, wherein the fabric sheet drapes down from the rollers and rests flat on the mattress in a center region of the mattress, wherein the rollers are configured to engage the fabric sheet and to rotate to pull the fabric sheet in an outward direction. When the fabric sheet is pulled taut, it forms a sloped profile to help push the user to turn to their sides.

In another aspect, the present invention provides a method of operating a bed, the bed comprising a mattress, a support frame, two rollers mounted on the support frame parallel to a longitudinal direction of the mattress and located near two edges of the mattress and above the top surface of the mattress, one or two motors, and a fabric sheet rolled on the two rollers and loosely suspended between the two rollers across the mattress, draping down from the rollers and rests flat on the mattress in a center region of the mattress, the method including: by an audio sensor, detecting a snoring sound of the user; and by a controller, in response to detecting the snoring sound of the user, controlling the one or two motors to rotate one of the two rollers to pull the fabric sheet in an outward direction.

It is to be understood that both the foregoing general description and the following detailed description are exemplary and explanatory and are intended to provide further explanation of the invention as claimed.

### BRIEF DESCRIPTION OF DRAWINGS

FIG. 1 schematically illustrates a bed equipped with an adjustable fabric sheet to facilitate change of the user's sleeping position according to an embodiment of the present invention.

FIG. 2 schematically illustrates the bed in FIG. 1 being operated to change the user's sleeping position.

FIG. 3 schematically illustrates a roller pulling a fabric sheet according to an embodiment of the present invention.

FIG. 4 schematically illustrates a roller pulling a fabric sheet according to another embodiment of the present invention.

FIG. 5 is a schematical block diagram illustrating a bed and its control system according to an embodiment of the present invention.

### DETAILED DESCRIPTION OF THE INVENTION

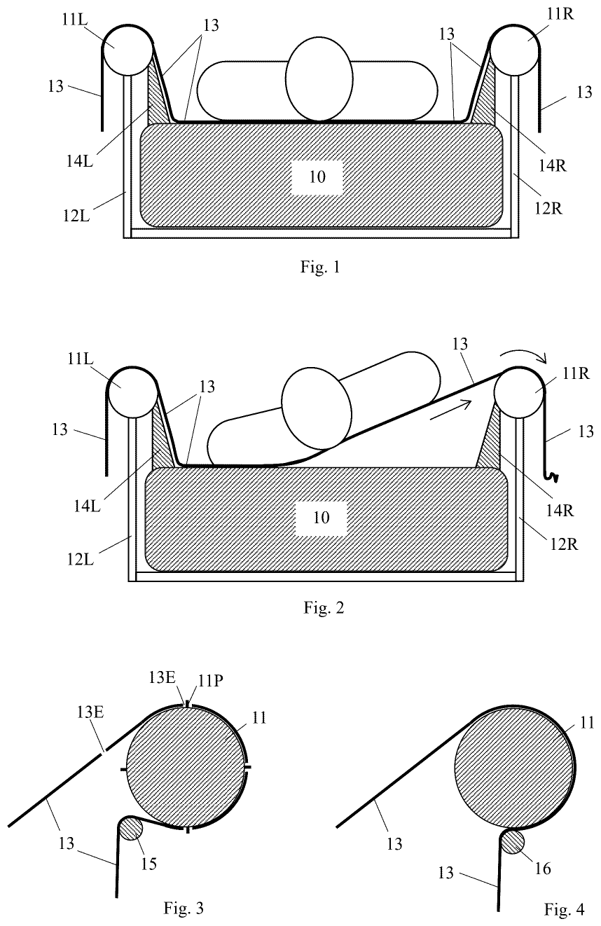

FIGS. 1 and 2 schematically illustrate a portion of a bed capable of changing the elevation profile of the sleeping surface to help the user change their sleeping position to reduce snoring.

As shown in FIG. 1, which is a side view taken along a longitudinal direction of the bed, the bed is equipped with two side rollers 11L and 11R oriented parallel to the longitudinal direction of the bed, located near the two side edges of the support member 10 (e.g., a mattress having any suitable structure) of the bed, above the bed surface. The rollers 11L and 11R are respectively supported by support frames 12L and 12R, which may be a part of a bed frame or side rails. A continuous fabric sheet 13 is rolled on and loosely suspended between the two rollers 11L and 11R across the bed, such that the sheet generally rests flat on the mattress surface 10 in the center region of the bed, and the two end portions drape downwardly from the rollers. The user normally sleeps in the flat center region of the bed as shown in FIG. 1.

When needed, one of the rollers is controlled to rotate and thereby pull the fabric sheet in one direction (in a direction away from the bed), causing the pulled section of the sheet to become taut and form a sloped profile, for example, as shown in FIG. 2 (same viewing direction as in FIG. 1). In this example, the roller 12 on the right rotates clockwise and pulls the fabric sheet toward the right as indicated by the two arrows in FIG. 2, causing the right hand portion of the sheet to become taut between the user and the roller and form a left-facing slope. This helps to gently push the user to turn to their left, preferably without waking them up. The shape of the fabric sheet is intended to provide an initial push, while the turn is typically completed by the user themselves. It is generally known that changing sleeping position may help stop snoring. After a predetermined period of time such as one minute, the roller is controlled to rotate in the opposite direction to release the tension in the fabric so that it lies flat on the bed surface again.

It should be understood that FIG. 2 schematically shows one state during the process of the sheet being pulled. From the initial state shown in FIG. 1, when the sheet starts to be pulled by the right roller 11R, the section between the right roller and the right shoulder of the user first become taut; as the roller 11R continues to pull the sheet, the right shoulder is gently lifted, and then a larger portion of the right side of the body is lifted; etc. It should also be noted that because the weight of the user is still partly resting directly on the mattress 10 (e.g. in the state shown in FIG. 2, the left shoulder is directly resting on the mattress), this tends to prevent the portion of the fabric sheet located between the user's body and the mattress from being dragged sideways across the bed; rather, the pulling of the fabric sheet by the roller primarily causes the gentle lifting of one side of the user's body.

The rollers 11L and 11R need not extend the entire length of the bed. In preferred embodiments, the rollers are about 300-400 mm long, and the fabric sheet is correspondingly 300-400 mm wide (in the longitudinal direction of the bed), located at a location corresponding to the middle and/or upper torso of the user. For example, the head end of the roller may be about 400-500 mm from the head end of the mattress. While the fabric sheet acts on only a portion of the user's body, it can provide sufficient action to initiate the turn which is expected to be completed by the user themselves. In alternative embodiments, the rollers may be longer and the fabric sheet may be located under a larger section of the user's torso and/or portions of the head (the pillow). As a general matter, shorter rollers can reduce overall cost of the bed system.

The useable width of the bed, defined by the distance between the two inner edges of the rollers, may be, for example, 500-700 mm, and the width of the mattress 10 (which has a flat and generally rectangular shaped top surface) is preferably similar to the bed width. The height of the top edge of the rollers is preferably 200-300 mm. The ratio of the height of the top edge of the roller to the width of the bed approximately determines the range of the sloping angle of the fabric sheet when it is pulled taut. The diameter of the rollers may be, for example, 100-200 mm. The suitable height of the rotational axes of the rollers may be determined based on the desired height of the top edge of the rollers and the diameter of the rollers.

Any suitable structure may be used for the support frame 12L and 12R. In preferred embodiments, the support frames 12L and 12R are moveable to lower the rollers 11L and 11R below the top surface of the mattress 10 when the user is not sleeping in the bed. This may be achieved using any suitable structures and mechanisms, such as those similar to the moveable side rails commonly provided on hospital beds. Soft cushions 14L and 14R may be provided inside the support frames 12L and 12R to protect the user.

The fabric sheet 13 is preferably made of a strong material, such as canvas or other suitable sheet materials, that can withstand the pulling force required to pull the sheet taut to form a sloped profile when the user is sleeping on it. In some embodiments, the fabric sheet can withstand a pulling force of at least 200 kg. The fabric sheet may also have a compound structure, i.e., be made of two sheets, with one underlying sheet providing structural strength and one top cover sheet made of a softer and more comfortable material. The top sheet may be removably attached to the underlying sheet.

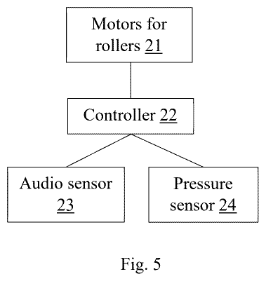

To facilitate the pulling of the fabric sheet by the rollers, in some embodiment, shown in FIG. 3, portions of the fabric sheet 13 that come in contact with the roller surface during the pulling process are provided with a number of small holes with eyelets 13A, and the surface of the roller 11 (11R or 11L) is correspondingly provided with short protruding pins 11P that fit in the eyelets. The engagement of the pins 11P and the eyelets 13E facilitates the pulling of the fabric sheet. To further increase the fulling force, an auxiliary roller 15 may be provided adjacent and parallel to the roller 11 to keep the fabric sheet 13 wrapped around a larger portion of the roller 11. Weights (not shown in the drawings) may also be provided at the two ends (left and right) of the fabric sheet 13 to keep the fabric sheet in contact with the rollers 11L and 11R. In some other embodiments, shown in FIG. 4, a tensioning roller 16 (e.g. a passive roller) is provided to apply a tension to the fabric sheet against the main roller 11. The fabric sheet 13 is pressed between rollers 11 and 16 which helps to keep the fabric sheet wound around the roller 11. The roller 11 may have a textured surface or fine teeth to increase the friction between the roller and the fabric sheet to facilitate pulling of the fabric sheet. Other suitable structures may alternatively be used to increase the engagement between the roller and the fabric sheet to facilitate pulling of the fabric sheet.

The rollers 11L and 11R are driven by motors. Details of the structures of the rollers, the support frame, the motors, and other mechanical and electrical components are not show and described here, as those of ordinary skill in the art can readily implement the rollers and related structures using known mechanical and electrical components.

FIG. 4 is a schematic block diagram showing various components of the bed and its control system. The components are communicatively coupled to each other by wired or wireless communication channels. In preferred embodiments, the rollers are operated by electric motors 21 which are controlled by a controller 22. The motors may be powered by battery or plug-in electricity. Preferably, the operation of the roller to change the profile of the fabric sheet is performed only if snoring is detected. To this end, an audio sensor 23 is provided adjacent to the bed to sense the snoring sound of the user. When the user is not snoring, the rollers are not operated and the fabric sheet stays flat as shown in FIG. 1. When snoring is detected by the audio sensor 12, one of the rollers is operated to pull the fabric sheet, thereby creating the sloped profile to help the user change their sleeping position.

Optionally, pressure sensors 24 may be provided at multiple points over the mattress 10 to detect the user's sleeping position (e.g., on their back or on their side, and which side). The information sensed by the pressure sensors 24 may be used by the controller 22 to control the operation of the rollers (once the sound sensor detects snoring), e.g., to determine whether the left roller or the right roller should be operated and how much fabric sheet should be pulled, etc.

The controller 22 may also be coupled to a user interface device (not shown), such as a control panel (e.g., a panel mounted on the side of the bed or on the wall of the bedroom), a handheld device (e.g., a remote control device, a smart phone, etc.), etc., which allows the user to interact with the controller. The controller 22 may include a microcontroller or any other suitable electronic circuits.

It will be apparent to those skilled in the art that various modification and variations can be made in the air mattress and related operation method of the present invention without departing from the spirit or scope of the invention. Thus, it is intended that the present invention cover modifications and variations that come within the scope of the appended claims and their equivalents.

1. A bed comprising:
a mattress having a flat top surface configured to receive a user's body;
a support frame;
two rollers mounted on the support frame, oriented parallel to a longitudinal direction of the mattress, located near two side edges of the mattress and above the top surface of the mattress;
one or two motors configured to drive the rollers to rotate independently of each other; and
a fabric sheet rolled on the two rollers and loosely suspended between the two rollers across the mattress, wherein the fabric sheet drapes down from the rollers and rests flat on the mattress in a center region of the mattress,
wherein the rollers are configured to engage the fabric sheet and to rotate to pull the fabric sheet in an outward direction.

2. The bed of claim 1, wherein the rollers are 300-400 mm in length and located at a longitudinal location corresponding to an upper torso of the user.

3. The bed of claim 1, wherein a distance between two inner edges of the rollers is 500-700 mm, and a height of a top edge of the rollers is 200-300 mm above the top surface of the mattress.

4. The bed of claim 1, wherein the fabric sheet is made of canvas.

5. The bed of claim 1, wherein portions of the fabric sheet include holes with eyelets, and wherein the rollers have protruding pins on their surfaces configured to engage the eyelets.

6. The bed of claim 1, further comprising two tensioning rollers configured to apply a tension to the fabric sheet against the corresponding rollers.

7. The bed of claim 1, further comprising soft cushions located inside of the support frame.

8. The bed of claim 1, wherein the support frame is configured to lower the two rollers to positions below the top surface of the mattress.

9. The bed of claim 1, further comprising a controller coupled to the one or two motors, and an audio sensor coupled to the controller and configured to sense a snoring sound of the user, wherein the controller is configured to control at least one of the rollers to rotate and pull the fabric sheet in response to the audio sensor detecting the snoring sound.

10. The bed of claim 1, further comprising a plurality of pressure sensors located at multiple points over the mattress, coupled to the controller, and configured to detect the user's sleeping position, wherein the controller is configured to control at least one of the rollers to rotate and pull the fabric sheet in response to the detected sleeping position.

11. A method of operating a bed, the bed comprising a mattress, a support frame, two rollers mounted on the support frame parallel to a longitudinal direction of the mattress and located near two edges of the mattress and above the top surface of the mattress, one or two motors, and a fabric sheet rolled on the two rollers and loosely suspended between the two rollers across the mattress, draping down from the rollers and rests flat on the mattress in a center region of the mattress, the method comprising:
by an audio sensor, detecting a snoring sound of the user; and
by a controller, in response to detecting the snoring sound of the user, controlling the one or two motors to rotate one of the two rollers to pull the fabric sheet in an outward direction.
